(19) United States
(12) Patent Application Publication
(10) Pub. No.: US 2020/0390831 A1
(43) Pub. Date: Dec. 17, 2020

(54) Probiotic

(71) Applicant: Jun Guo, Shenzhen (CN)

(72) Inventor: Jun Guo, Shenzhen (CN)

(21) Appl. No.: 16/442,464

(22) Filed: Jun. 15, 2019

(51) Int. Cl. A61K 35/747; A61K 35/745

(52) U.S. Cl. CPC A61K 35/747; A61K 35/745

(57) ABSTRACT

The present invention relates to a probiotic, including bacterial powder, comprehensive enzymes, tea polyphenols, soluble dietary fibers, oligofructoses and mannitols; the bacterial powder is composed of the following materials: Lactobacillusacidophilus LA1063, BifidobacteriumlongumBL986, Lactobacillus paracaseiLPC12, Lactobacillus rhamnosusLRH10, Lactobacillus fermentumLF26, Streptococcus thermophilusST30, and Lactobacillus helveticusLH43. Compared with the prior art, the present invention has the following beneficial effects: (1) having a long-lasting activity and being resistance to gastric acid; (2) being shelf-stable and processing-stable; and (3) having no side effects and having high safety.

### TECHNICAL FIELD

The present invention relates to a probiotic, and belongs to the technical field of probiotics.

### BACKGROUND

The probiotic is a kind of active microorganisms that are beneficial to the host. It is the generic term for active beneficial microorganisms that are colonized in the intestinal tract and reproductive system of human body and can produce exact health effects to improve the microecological balance of the host and play a beneficial role. The probiotic is a kind of living microorganisms that benefit the health of the host when a sufficient amount of the probiotic is uptaken by the host. Therefore, the active probiotic is often used as an auxiliary additive for a nutraceutical product. Beneficial bacteria or fungi in the body of human and animals mainly include clostridium butyricum, lactobacillus, bifidobacterium, actinomycete, and yeast. At present, the most functional product studied in the world is mainly composite active probiotics composed of the aforementioned various microorganisms which are widely used in the fields of bioengineering, industry and agriculture, food safety and life health.

### SUMMARY

In view of the defects of the prior art, an objective of the present invention is to provide a probiotic which combines probiotics and enzymes and has effects such as enhancing the immunity, balancing endocrine, promoting metabolism, protecting the intestinal tract, and promoting cell activating.

In order to achieve the aforementioned objective, the technical solution of the present invention is as follows.

A probiotic is made from the following raw materials: bacterial powder, comprehensive enzymes, tea polyphenols, soluble dietary fibers, oligofructoses and mannitols; where the bacterial powder is 30-50 parts by weight; the comprehensive enzymes are 20-40 parts by weight; the tea polyphenols are 10-30 parts by weight; the soluble dietary fibers are 2-10 parts by weight; the oligofructoses are 5-10 parts by weight; and the mannitols are 2-8 parts by weight.

Furthermore, the bacterial powder is composed of the following raw materials: Lactobacillusacidophilus LA1063, BifidobacteriumlongumBL986, Lactobacillus paracaseiLPC12, Lactobacillus rhamnosusLRH10, LactobacillusfermentumLF26, Streptococcus thermophilusST30, and Lactobacillus helveticusLH43.

Preferably, the mass ratio of respective raw materials in the bacterial powder is: Lactobacillusacidophilus LA1063: BifidobacteriumlongumBL986: Lactobacillus paracaseiLPC12:LactobacillusrhamnosusLRH10:Lactobacillus fermentumLF26: Streptococcus thermophilusST30:Lactobacillus helveticusLH43=1:(1-3):(1-4):(1-5): (1-4):(1-3):(1-2).

Furthermore, the comprehensive enzymes consist of the following components: Bromelain, Papain, cellulase, diastase, and lipase.

Preferably, the mass ratio of respective materials in the comprehensive enzymes is: Bromelain:Papain:cellulase:diastase:lipase=1:(1-2):(1-3):(1-2):(1-4).

In the probiotic, a 7-strain symbiotic fermentation technology is adopted, which is different from the single-strain fermentation in that the combinative strains grow together, each of which plays its role and helps each other, effectively improving the ecology of the intestinal bacteria. A multi-layer embedding technology is used, calcium ions (Ca⁺) are added to stabilize the cell membrane, such that the probiotic is shelf-stable; total milk proteins are employed to isolate oxygen, such that the probiotic is processing-stable; and colloids which are dissolved in an intestinal environment are added, such that the probiotic is resistant to gastric acid. The Multi-layer embedding technology can enhance the resistance of the probiotic to temperature, pressure, gastric acid and choline, improve the storage stability of the product, improve the processing stability of the product, and ensure the functional validity of the product.

The beneficial effect of the present invention is: compared with the prior art, the present invention has the following beneficial technical effects:

(1) having a long-lasting activity and being resistance to gastric acid, Cold chain drying and multi-layer embedding technologies are employed, such that this active probiotic has long-lasting activity and resistance to gastric acid, adapts to the intestinal environment, and can be effectively colonized in the intestinal tract, maximally maintaining its stability and activity.

(2) being shelf-stable and processing-stable, The probiotic is composed of 7 co-fermented strains, and is processing-stable since total milk proteins are employed to isolate oxygen; calcium ions are added to stabilize the cell membrane, such that the probiotic can adhere to the intestinal wall, be resident in the intestinal tract, thereby improving the working time and working ability of the probiotic.

(3) having no side effects and having high safety, Respective ingredients are Food ingredients that can be directly consumed without any side effect, and both the active probiotics and oligofructoses in the formula can inhibit the growth and proliferation of harmful bacteria, such that it is possible to achieve stable storage without additional addition of preservatives, thereby reducing toxic and side effects caused by the additive while achieving a good intestinal-clearing and detoxifying effect.

### DESCRIPTION OF THE EMBODIMENTS

The specific embodiments of the present invention are described below in conjunction with Examples, so as to provide a better understanding of the present invention.

### EXAMPLE 1

The probiotic of this Example included bacterial powder, comprehensive enzymes, tea polyphenols, soluble dietary fibers, oligofructoses and mannitols, the bacterial powder was composed of the following materials: Lactobacillusacidophilus LA1063, BifidobacteriumlongumBL986, Lactobacillus paracaseiLPC12, Lactobacillus rhamnosusLRH10, Lactobacillus fermentumLF26, Streptococcus thermophilusST30, and Lactobacillus helveticusLH43; and the comprehensive enzymes consisted of the following components: Bromelain, Papain, cellulase, diastase, and lipase.

The probiotic consisted of the following materials in parts by mass: 30 parts of the bacterial powder, 20 parts of the comprehensive enzymes, 10 parts of the tea polyphenols, 2 parts of the soluble dietary fibers, 5 parts of the oligofructoses, and 2 parts of the mannitols.

The mass ratio of respective materials in the bacterial powder was: Lactobacillusacidophilus LA1063:BifidobacteriumlongumBL986:Lactobacillus paracaseiLPC12:Lactobacillus rhamnosusLRH10:Lactobacillus fermentumLF26: Streptococcus thermophilusST30:Lactobacillus helveticusLH43 =1:3:2:4:3:2:1. The mass ratio of respective materials in the comprehensive enzymes was: Bromelain: Papain: cellulase: diastase: lipase=1:2:2:1:3.

In the probiotic, a 7-strain symbiotic fermentation technology was adopted, which was different from the single-strain fermentation in that the combinative strains grew together, each of which played its role and helped each other, effectively improving the ecology of the intestinal bacteria. A multi-layer embedding technology was used, calcium ions (Ca⁺) were added to stabilize the cell membrane, such that the probiotic was shelf-stable; total milk proteins were employed to isolate oxygen, such that the probiotic was processing-stable; and colloids which were dissolved in an intestinal environment were added, such that the probiotic was resistant to gastric acid. The Multi-layer embedding technology could enhance the resistance of the probiotic to temperature, pressure, gastric acid and choline, improve the storage stability of the product, improve the processing stability of the product, and ensure the functional validity of the product.

### EXAMPLE 2

The probiotic of this Example included bacterial powder, comprehensive enzymes, tea polyphenols, soluble dietary fibers, oligofructoses and mannitols, the bacterial powder was composed of the following materials: Lactobacillusacidophilus LA1063, BifidobacteriumlongumBL986, Lactobacillus paracaseiLPC12, Lactobacillus rhamnosusLRH10, Lactobacillus fermentumLF26, Streptococcus thermophilusST30, and Lactobacillus helveticusLH43; and the comprehensive enzymes consisted of the following components: Bromelain, Papain, cellulase, diastase, and lipase.

The probiotic consisted of the following materials in parts by mass: 50 parts of the bacterial powder, 40 parts of the comprehensive enzymes, 30 parts of the tea polyphenols, 10 parts of the soluble dietary fibers, 10 parts of the oligofructoses, and 8 parts of the mannitols.

The mass ratio of respective materials in the bacterial powder was : Lactobacillusacidophilus LA1063:BifidobacteriumlongumBL986:Lactobacillus paracaseiLPC12:Lactobacillus rhamnosusLRH10:Lactobacillus fermentumLF26: Streptococcus thermophilusST30:Lactobacillus helveticusLH43 =1:3:2:5:4:3:2.

The mass ratio of respective materials in the comprehensive enzymes was: Bromelain : Papain:cellulase:diastase:lipase=1:2:3:2:4.

In the probiotic, a 7-strain symbiotic fermentation technology was adopted, which was different from the single-strain fermentation in that the combinative strains grew together, each of which played its role and helped each other, effectively improving the ecology of the intestinal bacteria. A multi-layer embedding technology was used, calcium ions (Ca⁺) were added to stabilize the cell membrane, such that the probiotic was shelf-stable; total milk proteins were employed to isolate oxygen, such that the probiotic was processing-stable; and colloids which were dissolved in an intestinal environment were added, such that the probiotic was resistant to gastric acid. The Multi-layer embedding technology could enhance the resistance of the probiotic to temperature, pressure, gastric acid and choline, improve the storage stability of the product, improve the processing stability of the product, and ensure the functional validity of the product.

The foregoing descriptions are preferred implementations of the present invention. It should be noted that for those of ordinary skills in the art, several improvements and modifications may further be made without departing from the principle of the present invention. These improvements and modifications should also be deemed as falling within the protection scope of the present invention.

what is claimed is:

1. A probiotic, wherein the probiotic comprises bacterial powder, comprehensive enzymes, tea polyphenols, soluble dietary fibers, oligofructoses and mannitols, the bacterial powder is composed of the following materials: Lactobacillusacidophilus LA1063, BifidobacteriumlongumBL986, Lactobacillus paracaseiLPC12, Lactobacillus rhamnosusLRH10, Lactobacillus fermentumLF26, Streptococcus thermophilusST30, and Lactobacillus helveticusLH43; and the comprehensive enzymes consist of the following components: Bromelain, Papain, cellulase, diastase, and lipase.

2. The probiotic of claim 1, wherein the probiotic consists of the following materials in parts by mass: 30-50 parts of the bacterial powder, 20-40 parts of the comprehensive enzymes, 10-30 parts of the tea polyphenols, 2-10 parts of the soluble dietary fibers, 5-10 parts of the oligofructoses, and 2-8 parts of the mannitols.

3. The probiotic of claim 1, wherein the mass ratio of respective materials in the bacterial powder is: Lactobacillusacidophilus LA1063: BifidobacteriumlongumBL986: Lactobacillus paracaseiLPC12 : Lactobacillus rhamnosusLRH10: Lactobacillus fermentumLF26: Streptococcus thermophilusST30:Lactobacillus helveticusLH43=1:(1-3):(1-4):(1-5): (1-4):(1-3):(1-2).

4. The probiotic of claim 1, wherein the mass ratio of respective materials in the comprehensive enzymes is: Bromelain:Papain:cellulase:diastase:lipase=1:(1-2):(1-3): (1-2):(1-4).
